(19) United States
(12) Patent Application Publication
(10) Pub. No.: US 2026/0295292 A1
(43) Pub. Date: Oct. 1, 2026

(54) PHYSIOTHERAPY COMB AND METHOD OF ACTIVATING THE SAME

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Huang Jianlong, Shenzhen (CN); Tang Yuanzhou, Shenzhen (CN)

(73) Assignee: SHENZHEN NUON MEDICAL EQUIPMENT CO., LTD., Shenzhen (CN)

(21) Appl. No.: 19/277,479

(22) Filed: Jul. 23, 2025

(30) Foreign Application Priority Data
Mar. 28, 2025 (CN) 2025103884286
Mar. 28, 2025 (CN) 2025205809529

(51) Int. Cl. A61N 5/06; A61H 23/02; A61N 1/04; A61N 1/36

(52) U.S. Cl. CPC A61N 5/0617; A61H 23/02; A61N 1/0408; A61N 1/36014; A61H 2201/0153; A61H 2201/02; A61H 2201/105; A61H 2201/1207; A61H 2201/1604; A61H 2201/5025; A61H 2201/5058; A61H 2205/021; A61N 2005/0626; A61N 2005/0632; A61N 2005/0644; A61N 2005/0652; A61N 2005/0663

(57) ABSTRACT

A physiotherapy comb is disclosed. The physiotherapy comb comprises a connecting portion defining a storage chamber. A first switching element is disposed within the connecting portion. A comb body having a plurality of comb teeth and rotatably connected to the connecting portion about a rotational axis. The comb body may be movable between a first position and a second position with respect to the connecting portion. A physiotherapy element is disposed within the comb body. A circuit board is disposed within the comb body and electrically connected to the physiotherapy element. A second switching element is disposed within the comb body and is electrically connected to the circuit board.

### DESCRIPTION

### TECHNICAL FIELD

The present invention relates generally to the field of electro-mechanical personal care devices and more particularly to a physiotherapy comb and method of activating the same.

### BACKGROUND

Combs are one of the most commonly used personal care tools for grooming hair. People use combs every day to detangle and style their hair, and many also use them to apply hair care products like oils, conditioners, or scalp treatments. As personal grooming habits evolve, there is a growing interest in using devices that not only style hair but also provide additional health or therapeutic benefits for the scalp and hair. In response to this need, some modern combs have been developed with built-in features such as light therapy for scalp treatment, microcurrent stimulation for improving blood flow, and vibration for massaging the scalp. These types of physiotherapy combs offer combined grooming and wellness functions in a single device.

However, most of these physiotherapy combs rely on button-based activation to turn their therapy features on or off. This can cause several problems. First, buttons can be pressed accidentally while carrying the comb in a bag or during use, which may drain the battery or cause unwanted stimulation. Second, users may forget to turn off the device, leading to unnecessary power consumption. Third, many such devices are not designed to dispense liquid treatments efficiently while combing, requiring a separate step for applying hair care solutions.

Therefore, there is a need for a physiotherapy comb that can automatically activate or deactivate its therapy functions based on its physical position, without requiring manual button presses. There is also a need for a comb that can dispense liquid treatments during use, making the grooming process more efficient and convenient for users.

### OBJECTIVES OF THE INVENTION

An objective of the present invention is to provide a physiotherapy comb that integrates therapeutic functionality such as such as phototherapy, microcurrent, iontophoresis, massage, hot or cold sensation, through components like a lamp board and light-emitting diodes (LEDs), heating element, cooling element, microcurrent element etc., thereby enhancing the scalp care experience during grooming.

Another objective of the present invention is to provide a physiotherapy comb that activates and deactivates therapeutic functions automatically based on the relative position of the comb body with respect to a connecting portion, thereby eliminating the need for manual switching and reducing the risk of accidental activation.

Another objective of the present invention is to provide a physiotherapy comb equipped with a mechanical safety mechanism that restricts unintended rotation or use of the comb body, enhancing user safety and control during operation.

Another objective of the present invention is to provide a physiotherapy comb that includes an integrated liquid reservoir and dispensing mechanism to apply treatment solutions to the scalp simultaneously during combing.

Yet another objective of the present invention is to offer a compact and modular design wherein all functional components, including physiotherapy elements, switching mechanisms, and power circuits, are effectively housed within the comb body and connecting portion.

A further objective of the present invention is to improve the hygiene of the comb during storage by integrating a UV disinfection element that is automatically activated when the comb is folded, effectively sanitizing liquid-contact components and comb teeth.

An additional objective of the present invention is to utilize infrared, red, blue, or ultraviolet LEDs selected for therapeutic and antimicrobial properties, thereby offering tailored scalp treatments and enhancing device hygiene through scientifically proven light frequencies.

An additional objective of the present invention is to provide a position-sensitive switching mechanism, such as a Hall effect sensor and magnet or a mechanical travel switch, that simplifies user interaction by eliminating the need for dedicated external buttons.

It is also an objective of the present invention to provide a physiotherapy comb with a charging system embedded in a rotating shaft, allowing a convenient power supply without compromising the compactness or structural integrity of the device.

### SUMMARY OF THE INVENTION

In an embodiment, a physiotherapy comb is disclosed. The physiotherapy comb may include a connecting portion defining a storage chamber. The physiotherapy comb may further include a comb body having a plurality of comb teeth and rotatably connected to the connecting portion about a rotational axis. In an embodiment, the comb body may be movable between a first position and a second position with respect to the connecting portion. The physiotherapy comb may further include a physiotherapy element disposed within the comb body. The physiotherapy comb further includes a circuit board disposed within the comb body and electrically connected to the physiotherapy element. In an embodiment, when the comb body is moved to the first position, the physiotherapy element is activated.

In accordance with the embodiment, the physiotherapy comb further includes a first switching element disposed within the connecting portion. The physiotherapy comb further includes a second switching element disposed within the comb body and electrically connected to the circuit board. In an embodiment, the first switching element and the second switching element are positioned in a manner such that, in the first position, the first switching element and the second switching element are aligned to allow activation of the physiotherapy element. In an embodiment, in the second position, the first switching element and the second switching element are spaced apart to disable the physiotherapy element.

In accordance with the embodiment, the physiotherapy comb further includes a push-actuated release mechanism and a limiting component. In an embodiment, the limiting component is movably engaged with a plug-in section of the connecting portion and gets disengaged upon actuation of the push-actuated release mechanism.

In accordance with the embodiment, the plurality of comb teeth are exposed in the first position and retracted into the storage chamber in the second position.

In accordance with the embodiment, the physiotherapy element includes a light therapy lamp that comprises at least one light-emitting diode (LED) selected from red, blue, or violet spectrum emitters.

In accordance with the embodiment, the physiotherapy element includes a pair of microcurrent electrodes disposed adjacent to or integrated with the plurality of comb teeth.

In accordance with the embodiment, the physiotherapy element includes a vibration motor configured to generate therapeutic vibrations transmitted through the comb body.

In accordance with the embodiment, the physiotherapy comb further includes a liquid reservoir disposed within a reservoir housing of the comb body and a liquid guide column having at least one fluid passage in fluid communication with the liquid reservoir. In an embodiment, the liquid guide column is positioned adjacent to the plurality of comb teeth.

In accordance with the embodiment, the first switching element includes a magnet, and the second switching element includes a Hall effect sensor configured to detect alignment of the first switching element and the second switching element for activating the physiotherapy element.

In accordance with the embodiment, the comb body includes a first casing and a second casing enclosing an installation space. In an embodiment, the installation space houses a mounting bracket, the physiotherapy element, and the circuit board. In an embodiment, a light-transmission base plate has integrally formed comb teeth attached to the first casing.

In accordance with the embodiment, the connecting portion includes a rotating shaft having a mounting through-hole, and an embedded charging electrode disposed within the mounting through-hole. In an embodiment, the embedded charging electrode is configured to supply power from an external source to the circuit board.

In another embodiment, a physiotherapy comb is disclosed. The physiotherapy comb includes a connecting portion defining a storage chamber. The physiotherapy comb further includes a first switching element disposed within the connecting portion. The physiotherapy comb further includes a comb body having a plurality of comb teeth and rotatably connected to the connecting portion about a rotational axis. In an embodiment, the comb body is movable between a first position and a second position with respect to the connecting portion. The physiotherapy comb further includes a physiotherapy element disposed within the comb body. The physiotherapy comb further includes a push-actuated release mechanism and a limiting component. In an embodiment, the limiting component is movably engaged with a plug-in section of the connecting portion to restrict rotation of the comb body relative to the connecting portion and gets disengaged upon actuation of the push-actuated release mechanism. The physiotherapy comb further includes a circuit board disposed within the comb body and electrically connected to the circuit board. In an embodiment, the first switching element and the second switching element are positioned in a manner such that, in the first position, the first switching element and the second switching element are aligned to allow activation of the physiotherapy element. In an embodiment, in the second position, the first switching element and the second switching element are spaced apart to disable the physiotherapy element.

In accordance with the embodiment, the plurality of comb teeth are exposed in the first position and retracted into the storage chamber in the second position.

In accordance with the embodiment, the physiotherapy element includes a light therapy lamp comprising at least one light-emitting diode (LED) selected from red, blue, or violet spectrum emitters.

In another embodiment, a method of activating a physiotherapy comb via mechanical motion. The method comprises rotating a comb body, having a plurality of comb teeth, relative to a connecting portion about a rotational axis from a second position to a first position. The method further includes aligning a first switching element, disposed within the connecting portion, with a second switching element, disposed within the comb body, to allow activation of a physiotherapy element. In an embodiment, the physiotherapy comb includes the connecting portion defining a storage chamber and may include the first switching element. The physiotherapy comb further includes the comb body having a plurality of comb teeth and rotatably connected to the connecting portion about the rotational axis, and movable between the first position and the second position. The physiotherapy comb further includes the physiotherapy element disposed within the comb body and configured to perform at least one of: phototherapy, microcurrent simulation, or vibration therapy. The physiotherapy comb may further include a circuit board disposed within the comb body and electrically connected to the physiotherapy element. The physiotherapy comb further includes the second switching element, electrically connected to the circuit board. In an embodiment, in the first position, the first switching element and the second switching element are aligned to allow activation of the physiotherapy element. In an embodiment, in the second position, the first switching element and the second switching element are spaced apart to disable the physiotherapy element.

In accordance with the embodiment, rotating the comb body to the first position includes exposing the plurality of comb teeth from within the storage chamber for combing hair while activating the physiotherapy element.

In accordance with the embodiment, the activation of the physiotherapy element includes energizing a light therapy lamp from a plug-in section of the connecting portion, thereby allowing rotation of the comb body from the second position to the first position.

In some embodiments, the method further includes transitioning the physiotherapy comb state from the first position (open state) to the second position (closed state), to automatically activate a UV light source for a predefined duration. The UV light source may disinfect the comb teeth and liquid-contact areas during storage, thereby reducing microbial contamination and enhancing hygiene.

In accordance with the embodiment, the method further includes engaging a push-actuated release mechanism to disengage a limiting component from a plug-in section of the connecting portion, thereby allowing rotation of the comb body from the second position to the first position.

In accordance with the embodiment, the first switching element includes a magnet, and the second switching element includes a Hall effect sensor configured to detect alignment of the first switching element and the second switching element for aligning the physiotherapy element.

The technical solution provided by the present invention involves configuring the comb body to be rotatably connected to the connecting portion and movable between a first position and a second position. When the comb body is rotated to the first position relative to the connecting portion, a first switching element disposed within the connecting portion is aligned with a second switching element disposed within the comb body. The alignment of the switching elements triggers a signal to a circuit board disposed within the comb body, which in turn activates a physiotherapy element such as a light therapy lamp, microcurrent electrode, or vibration motor. This allows the user to perform scalp physiotherapy while combing.

When the comb body is rotated back to the second position, the first switching element and the second switching element become spaced apart, resulting in the second switching element generating a deactivation signal to the circuit board, which then disables the physiotherapy element. Because the activation and deactivation of the physiotherapy function are entirely dependent on the rotational position of the comb body. The invention eliminates the need for external buttons and reduces the risk of accidental activation during storage or transport.

In one embodiment of the invention, the physiotherapy element performs at least one of: phototherapy, micro-current therapy, piezoelectric massage, ultrasonic wave therapy, thermal stimulation, or vibration therapy.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term "LED" refers to one or more light-emitting diode (LED) elements that are electrically connected and configured to emit light of specific wavelengths (including visible to IR wavelengths) suitable for therapeutic purposes. The LED module or element may include drive circuitry, heat dissipation structures, and optical elements such as lenses or diffusers to control light distribution.

In the context of this specification, terms like “light,” “radiation,” “irradiation,” “emission” and “illumination,” etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorised into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source,” etc., refers to a source emitting coherent laser light, or light-emitting diodes (“LEDs”). The term “light therapy” refers to light generated from any of the sources, such as lasers, LED sources, or Super luminous diodes (“SLD”) or Organic light-emitting diodes (OLED). In certain embodiments, the light source may include UV LEDs for non-therapeutic disinfection purposes when the comb is in a folded or storage state.

In the context of the specification, the term "occlusion piece" refers to a movable component, such as a valve or spring-loaded element, disposed at the outlet of the liquid guide column and configured to block or permit fluid flow based on contact with the scalp or user-applied pressure.

In the context of the specification, the term "lamp board" refers to a printed circuit board or similar substrate on which one or more light sources are mounted and electrically connected to a power source, such as a rechargeable battery.

In the context of the specification, the term "light source" refers to any active component, such as a light-emitting diode (LED), capable of emitting phototherapy light (e.g., red, infrared, or blue) toward the scalp.

In the context of the specification, the term "light transmission hole" refers to an aperture formed in the housing of the second rotating arm, aligned with the light source to allow therapeutic light to exit the comb and reach the scalp.

In the context of the specification, the term "light transmission plate" refers to a transparent or translucent cover positioned over the light transmission hole to protect internal components while allowing light to pass through.

In the context of the specification, the term "pressing switch" refers to a mechanical actuator disposed on the surface of the comb and configured to trigger an internal switching device that activates the lamp board or associated electronics.

In the context of the specification, the term “plurality” means two or more than two, unless otherwise indicated.

In the context of the specification, the term "several" means more than one, unless otherwise specified.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection elements integrated into the multifunctional comb.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc., in pure form or doped with elements such as Aluminium and Indium etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the case of a physiotherapy element being an electrode, the physiotherapy element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user. The electrodes are placed at or near nerves where the pain is located or at certain known trigger points.

### BRIEF DESCRIPTION OF THE DRAWING

The accompanying drawings, which are incorporated in and constitute a part of this disclosure, illustrate exemplary embodiments and, together with the description, serve to explain the disclosed principles.

To more clearly illustrate the technical solutions described in the embodiments of the present invention, the accompanying drawings referenced below are provided as part of the detailed description. It should be understood that these drawings represent only certain exemplary embodiments of the invention, and that other configurations and variations may be derived from these illustrations by those skilled in the art without departing from the scope of the present disclosure.

FIG. 1 illustrates a schematic diagram of a physiotherapy comb, wherein a comb body of the physiotherapy comb is in a first position, in accordance with an embodiment of the present disclosure.

FIG. 2 illustrates a schematic diagram of the physiotherapy comb, wherein the comb body is at a position between the first position and a second position, in accordance with an embodiment of the present disclosure.

FIG. 3 illustrates a schematic diagram of the physiotherapy comb, wherein the comb body is in the second position, in accordance with an embodiment of the present disclosure.

FIG. 4 illustrates an exploded view of the physiotherapy comb, in accordance with an embodiment of the present disclosure.

FIG. 5 illustrates a schematic diagram of a plane of the physiotherapy comb in FIG. 1, in accordance with an embodiment of the present disclosure.

FIG. 6 illustrates a cross-sectional view of a section of the physiotherapy comb at A-A in FIG. 5, in accordance with an embodiment of the present disclosure.

FIG. 7 illustrates a cross-sectional view of a section of the physiotherapy comb at B-B in FIG. 5, in accordance with an embodiment of the present disclosure.

FIG. 8 illustrates an exploded view of the comb body in FIG. 1, in accordance with an embodiment of the present disclosure.

FIG. 9 illustrates an exploded view of a connecting portion in FIG. 1, in accordance with an embodiment of the present disclosure.

FIG. 10 illustrates a schematic diagram of a part structure of the physiotherapy comb in FIG. 1, in accordance with an embodiment of the present disclosure.

FIG. 11 illustrates a schematic diagram of the structure of a rotating shaft in FIG. 9, in accordance with an embodiment of the present disclosure.

FIG. 12 illustrates a schematic diagram of a part structure of the physiotherapy comb in FIG. 1, in accordance with an embodiment of the present disclosure.

FIG. 13 illustrates a schematic diagram of a rotational mechanism of the physiotherapy comb, in accordance with another embodiment of the present disclosure.

FIG. 14 illustrates a schematic diagram of a sliding mechanism of the physiotherapy comb, in accordance with another embodiment of the present disclosure.

FIG. 15 is a flowchart of a methodology to activate the physiotherapy comb, in accordance with an embodiment of the present disclosure.

### DETAILED DESCRIPTION OF THE DRAWINGS

The foregoing description has broadly outlined the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific embodiments disclosed may be readily utilized as a basis for modifying other devices, systems, assemblies, and mechanisms for conducting the same purposes of the present disclosure. It should also be realized by those skilled in the art that such equivalent constructions do not depart from the scope of the disclosure as set forth in the appended claims. The novel features, which are believed to be characteristics of the disclosure, of its device or system, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

The technical solutions described in the embodiments of the present disclosure will be clearly and fully explained below with reference to the accompanying drawings. It should be understood that the embodiments provided herein are merely illustrative and do not represent the entirety of possible implementations. Variations and modifications to these embodiments that may be conceived by those skilled in the art, without departing from the spirit and scope of the invention and without the exercise of inventive faculty, are intended to fall within the scope of protection afforded by the present disclosure.

It should be noted that, in the description of the present invention, the terms "first," "second," and the like are used solely for the purpose of distinguishing between different elements and should not be construed as indicating any order, priority, or relative importance. Such terms are not intended to imply a specific sequence or quantity unless explicitly stated. Accordingly, references to “first” and “second” components may each refer to one or more of such components. Furthermore, the term "and/or" as used herein is intended to encompass any and all combinations of one or more of the associated listed items. For example, the expression “A and/or B” should be interpreted to include: A alone, B alone, or both A and B together.

The terms “including”, “comprises”, “comprising”, “comprising of” or any other variations thereof, are intended to cover a non-exclusive inclusion, such that a system or a device that comprises a list of components or steps does not include only those components or steps but may include other components or steps not expressly listed or inherent to such setup or device. In other words, one or more elements in a system or apparatus preceded by “comprises… a” do not, without more constraints, preclude the existence of other elements or additional elements in the system or apparatus.

Embodiments of the present invention provide a physiotherapy comb configured with integrated therapeutic components such as light therapy LEDs, microcurrent electrodes, a vibration motor, etc. The physiotherapy comb activates these functions based on the rotational position of the comb body relative to a connecting portion, enhancing user convenience and safety.

In an embodiment, the present invention provides a physiotherapy comb comprising a rotatable comb body and a connecting portion that houses various functional components. The comb body includes a plurality of comb teeth formed integrally on a light-transmitting base plate and is configured to rotate about a defined axis relative to the connecting portion. Disposed within the comb body are essential components such as a physiotherapy element (which may include LEDs, vibration motors, magnetic elements, ultrasonic wave therapy elements, piezoelectric elements, microcurrent electrodes, or heating/cooling elements), a circuit board, a liquid reservoir with a dispensing mechanism, and one or more switching elements. The connecting portion includes a storage chamber, a rotating shaft with embedded charging electrodes, and a complementary switching element. The alignment of switching elements determines the operational state of the physiotherapy components, ensuring that the therapeutic functionality activates only when the comb body is in the appropriate position. Additional features such as a push-actuated release mechanism, a limiting component to restrict unintended movement, and UV-based sterilization elements enhance both functionality and safety.

In another embodiment, a method is disclosed for activating the physiotherapy comb through mechanical manipulation. The method includes rotating the comb body from a second position, where the comb teeth are concealed within the storage chamber, to a first position, where the comb teeth are exposed for use. As the comb body is rotated, a first switching element disposed in the connecting portion aligns with a second switching element within the comb body. This alignment generates an electrical signal that activates the physiotherapy element embedded in the comb body. Upon returning the comb to its second position, the switching elements become misaligned, thereby deactivating the therapy function. This method not only eliminates the need for external buttons but also ensures intuitive, position-based control of the device, improving user experience and energy efficiency.

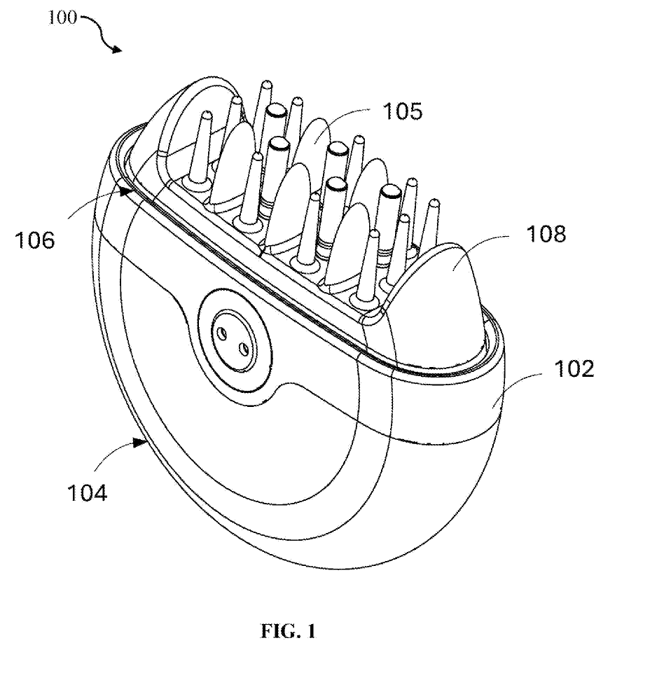

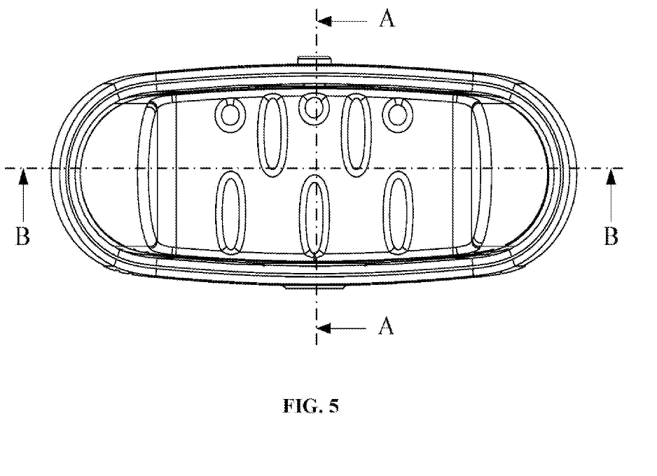

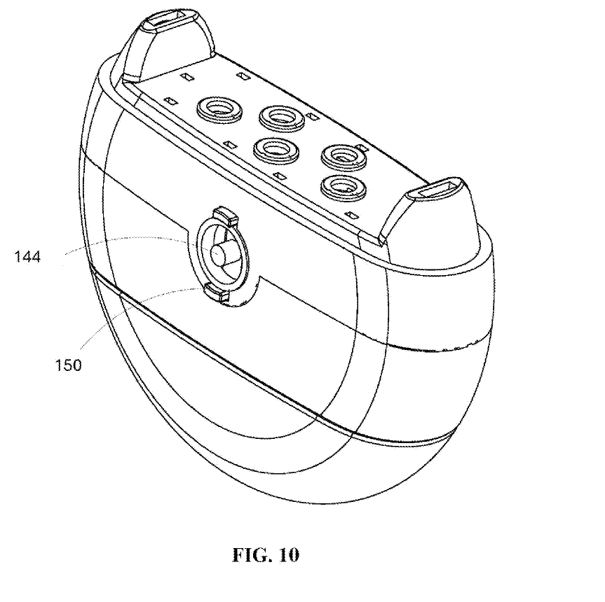

Reference will now be made to the exemplary embodiments of the disclosure, as illustrated in the accompanying drawings. Wherever possible, the same numerals have been used to refer to the same or like parts. The following paragraphs describe the present disclosure with reference to FIGS. 1-15. As summarized above, in one broad aspect, the present invention provides a physiotherapy comb and a method of activating the same.

Referring to FIGS. 1-8, the physiotherapy comb 100 comprises a connecting portion 102, a comb body 108, a physiotherapy element 194, a circuit board 136, and a switching piece 115. The comb body 108 includes a plurality of comb teeth 110. The physiotherapy element 194 and the circuit board 136 are arranged within the comb body 108, and the circuit board 136 is electrically connected to the physiotherapy element 194.

The comb body 108 is configured to be movably connected with the connecting portion 102 such that the comb body 108 assumes a first position and a second position relative to the connecting portion 102. The switching piece 115 includes a first switching element 116 disposed within the connecting portion 102 and a second switching element 117 disposed within the comb body 108. The second switching element 117 is electrically connected to the circuit board 136. When the comb body 108 is at the first position, the first switching element 116 and the second switching element 117 are aligned with each other or in contact, thereby enabling activation of the physiotherapy element 194. In an embodiment, the physiotherapy element 194 is selected from a group consisting of, but is not limited to a light-emitting diodes (LEDs), lasers, heating or cooling elements, Peltier devices, electrodes, pulsed magnetic fields (PMF), ultrasonic transducers, magnetic stimulators, microcurrent stimulators, electrical muscle stimulation (EMS) units, and vibration motors. When the comb body 108 is moved to the second position, the first switching element 116 and the second switching element 117 are spaced apart from one another, causing the physiotherapy element 194 to be deactivated.

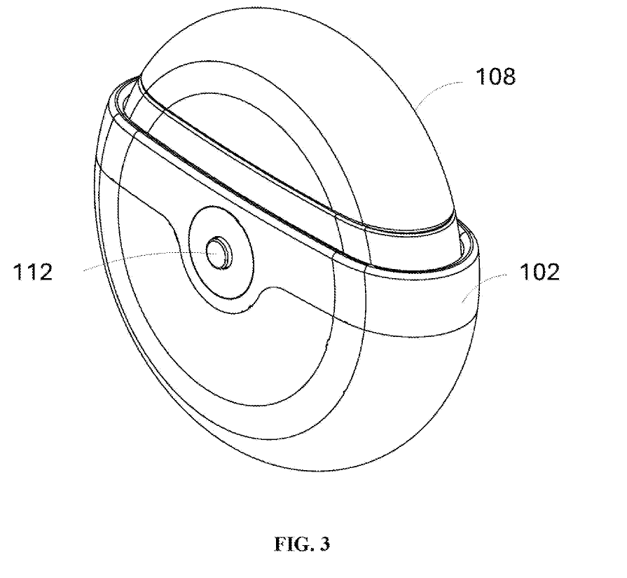

In the embodiment shown in FIG. 1, when the comb body 108 is moved to the first position relative to the connecting portion 102, the first switching element 116 located in the connecting portion 102 comes into alignment with, or abuts against, the second switching element 117 within the comb body. The positional change triggers the second switching element 117 to send a start-up signal to the circuit board 136. Upon receiving this signal, the circuit board 136 activates the physiotherapy element 194, allowing the user to perform scalp physiotherapy while using the physiotherapy comb 100. When the comb body 108 is rotated or moved to the second position relative to the connecting portion 102 (as depicted in FIG. 3), the first switching element 116 and the second switching element 117 are separated from each other, and the second switching element 117 generates a shutdown signal. This signal is sent to the circuit board 136, which in turn deactivates the physiotherapy element 194.

Because the activation of the physiotherapy element 194 is dependent solely on the relative position of the comb body 108, the physiotherapy comb 100 eliminates the need for manual power buttons. This mechanism effectively prevents unintended activation caused by accidental touches, particularly during handling, transport, or storage, and thereby enhances operational safety and convenience.

In an embodiment of the present disclosure, when the comb body 108 is in the first position, the plurality of comb teeth 110 are exposed for normal grooming operations. In the second position, the plurality of comb teeth 110 are retracted or covered by the connecting portion 102, such that the comb teeth 110 are partially or completely enclosed within the connecting portion 102. This arrangement helps to protect the comb teeth 110 from damage due to external contact when not in use. Moreover, the exposure of the comb teeth 110 in the first position automatically activates the physiotherapy function, allowing the user to simultaneously comb hair and receive therapeutic treatment. When the comb teeth 110 are in the retracted state, the physiotherapy function is automatically turned off.

The comb body 108 and the connecting portion 102 are movably connected in various ways, including but not limited to a rotary connection or a sliding connection, depending on the design implementation. The structural configuration of the comb body 108 and the connecting portion 102 may vary across embodiments. In some implementations, the connecting portion 102 defines a storage chamber 132, and the comb body 108 is rotatably connected to the connecting portion 102. In the first position, the comb teeth 110 extend outward from the storage chamber 132, allowing the physiotherapy comb 100 to be used for grooming. In the second position, the comb teeth 110 are retracted within the storage chamber 132, thereby reducing the profile of the physiotherapy comb 100 and protecting the comb teeth 110 when the physiotherapy comb 100 is not needed.

In alternate embodiments of the present disclosure, the physiotherapy comb 100 includes different structural arrangements for the connection between the comb body 108 and the connecting portion 102 to achieve movement between the first position and the second position.

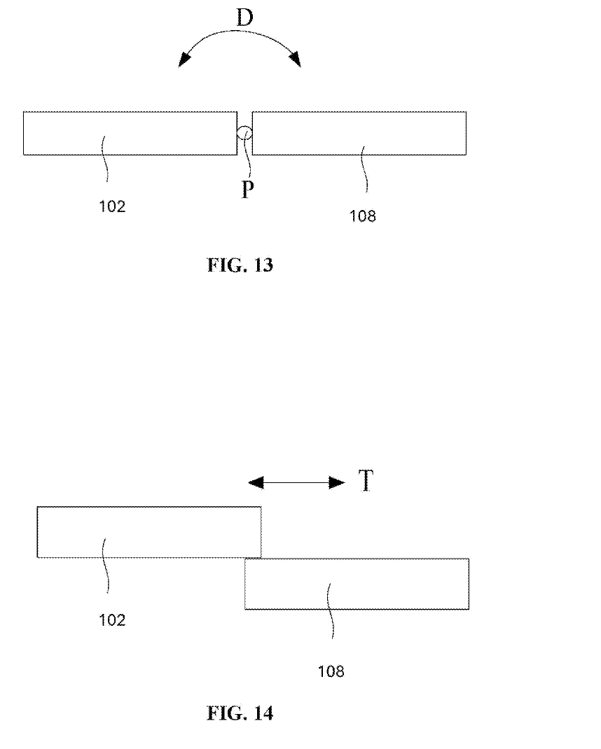

Referring to FIG. 13, in some embodiments, the comb body 108 and the connecting portion 102 are in an elongated shape. One end of the comb body 108 is rotatably connected to one end of the connecting portion 102 such that the two components are either folded or opened relative to each other about a central rotational axis (P). The direction of rotation (D) defines the axis along which the folding occurs. When the comb body 108 is folded toward the connecting portion 102, the physiotherapy comb is in the second position, wherein the comb teeth are concealed or enclosed. When the comb body 108 is fully opened relative to the connecting portion 102, the device assumes the first position, allowing the comb teeth to be exposed for grooming and activation of the physiotherapy element.

Referring to FIG. 14, in another embodiment, the comb body 108 and the connecting portion 102 are both an elongated shape and are configured in a manner such that their respective longitudinal axes are parallel. In this configuration, the comb body 108 is movably connected to the connecting portion 102 via a sliding mechanism. The sliding occurs along the lengthwise direction (T) of the comb body 108. When the comb body 108 slides to a position farthest from the connecting portion 102, the physiotherapy comb reaches its first position, wherein the overall length of the physiotherapy comb is extended, and the comb teeth are fully exposed for grooming and physiotherapy. Conversely, when the comb body 108 is slid to align and overlap with the connecting portion 102, the physiotherapy comb enters the second position, wherein the comb body 108 and the connecting portion 102 are substantially superimposed, thereby reducing the overall length of the physiotherapy comb. In this second position, the comb teeth are enclosed or protected, and the physiotherapy function is deactivated.

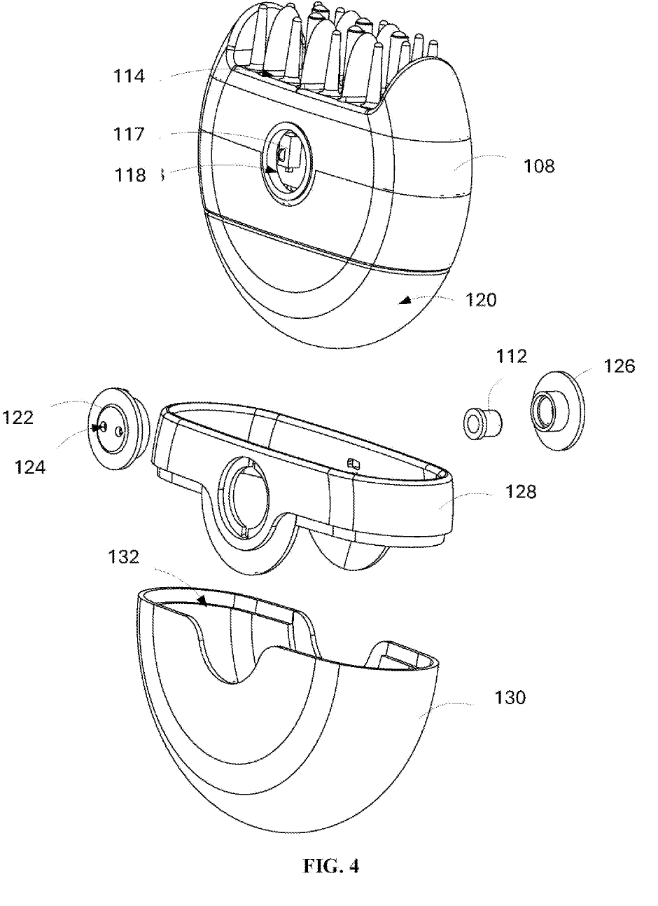

Referring to FIGS. 1 and 4, the storage chamber 132 is formed internally such that the comb teeth 110 of the comb body 108 are selectively retracted into or extended from the storage chamber 132 based on the position of the comb body 108. In certain embodiments, the storage chamber 132 further includes an ultraviolet (UV) light source. The UV light source serves a dual purpose, such as pre-activating a precursor composition stored in proximity to the comb teeth 110 or liquid guide column 186, and disinfecting and sterilizing the comb teeth 110 and internal surfaces of the storage chamber 132 when the comb body 108 is in the second position. This UV-based sterilization mechanism enhances hygienic use and facilitates the controlled activation of any photosensitive formulations stored within the physiotherapy comb 100.

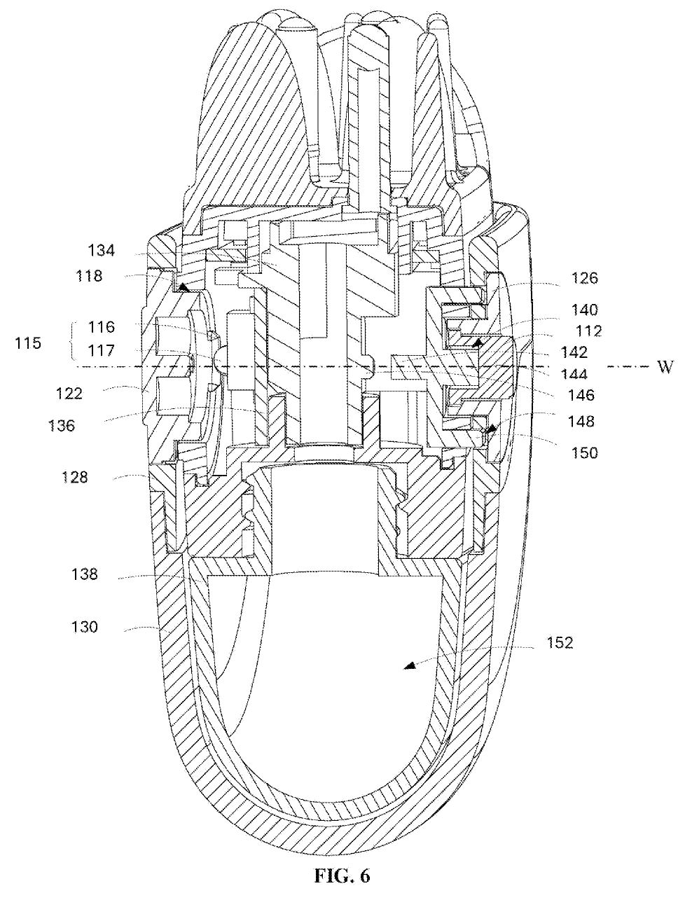

In some embodiments, the outer contour of the connecting portion 102 includes an arc edge 104 and a straight edge 106, the two edges being connected end to end from a semi-cylindrical or arc-segmented housing. The storage chamber 132 includes an open outlet located along the straight edge 106, and the center of curvature of the arc edge 104 aligns with the rotational axis W shared by the comb body 108 and the connecting portion 102 (as shown in FIG. 6). This configuration allows the comb body 108 to rotate within the storage chamber 132.

When the comb body 108 is rotated such that the comb teeth 110 protrude outward through the open side of the straight edge 106, the comb body 108 is in the first position, enabling grooming and automatic activation of the physiotherapy element 194 via the alignment of the first switching element 116 and second switching element 117. In this first position, the first switching element 116 and the second switching element 117 are brought into alignment. In one configuration, this alignment automatically activates the physiotherapy element 194 through proximity sensing or physical contact. In an alternative configuration, the alignment of the switching elements enables the physiotherapy element 194 to be manually activated by a push button disposed on the comb body 108 or in communication with the circuit board 136. When the comb body 108 is rotated inward such that the comb teeth 110 face the interior wall of the storage chamber 132, the comb body 108 is in the second position, wherein the comb teeth 110 are protected and the physiotherapy function is disabled.

The inclusion of the storage chamber 132 allows the comb body 108 to be at least partially enclosed within the connecting portion 102, resulting in a more compact form factor, which facilitates convenient storage and portability. Furthermore, the relative arrangement of the connecting portion 102 and comb body 108 permits rotational folding, wherein a larger overlapping area between the components further contributes to reducing the overall volume of the physiotherapy comb 100 when not in use.

In certain embodiments, the arc edge 104 of the connecting portion 102 is in the form of a precise or optimal arc, depending on aesthetic or functional design constraints. Likewise, the shape of the comb body 108 is adapted to correspond with the connecting portion 102. For instance, in some configurations, both the comb body 108 and the connecting portion 102 may be generally circular. In such cases, the outer contour of the comb body 108 may include an arc wall 120, with its ends joined by a connecting wall 114, which may be linear or contoured in design. The connecting wall 114 may support the attachment of the comb teeth 110. The arc wall 120 may also follow an optimized curvature, and it may be concentrically aligned with the arc edge 104 of the connecting portion 102 for smooth rotational motion.

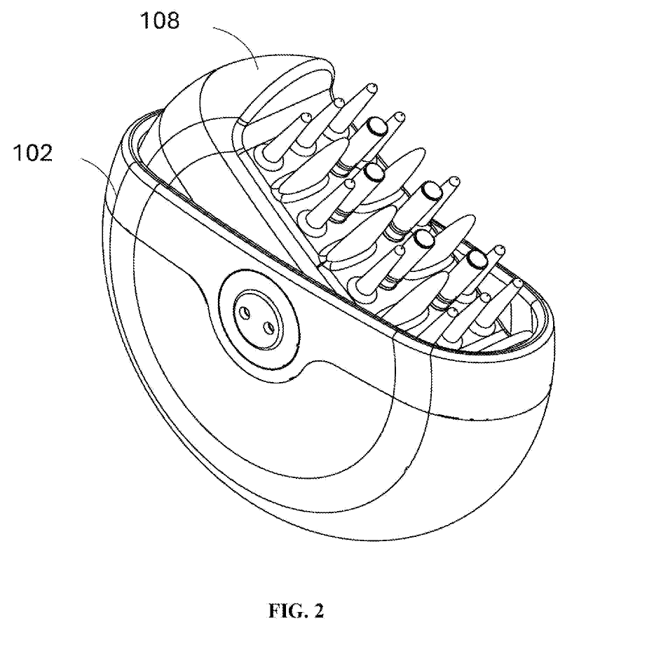

In some embodiments, the rotational angle between the first position and the second position is approximately 180 degrees, such that the transition between the two positions corresponds to a half-turn of the comb body 108 relative to the connecting portion 102, as depicted from FIGS. 1-3. FIG. 2 illustrates an intermediate position during this rotation.

In alternative embodiments, the precise position of activation is defined such that any configuration in which the comb teeth 110 are fully exposed outside the storage chamber 132 constitutes the first position, while any other configuration is treated as the second position. This allows for position-based activation of the physiotherapy function only when the physiotherapy comb 100 is in use.

Referring to FIGS. 6 and 8-10, in order to further avoid unintentional operation and prevent the comb body 108 from rotating freely with respect to the connecting portion 102, the physiotherapy comb 100 further includes a push-actuated release mechanism 112 and a limiting component 196. The push-actuated release mechanism 112 is movably connected to the connecting portion 102, and the limiting component 196 is movably connected to the comb body 108. In its default state, the limiting component 196 engages with a plug-in section 150 formed in the connecting portion 102, thereby restricting rotational movement of the comb body 108 relative to the connecting portion 102.

When the push-actuated release mechanism 112 is subjected to an external force, the push-actuated release mechanism 112 displaces the limiting component 196 such that the push-actuated release mechanism 112 disengages from the plug-in section 150. Specifically, as illustrated in FIG. 6, when no external force is applied to the push-actuated release mechanism 112, the limiting component 196 remains clamped within the connecting portion 102, thereby preventing the comb body 108 from rotating. Upon manual actuation, such as pressing, sliding, or turning, the push-actuated release mechanism 112 drives the limiting component 196 to withdraw from the clamping engagement, allowing the comb body 108 to rotate relative to the connecting portion 102.

In various embodiments, the push-actuated release mechanism 112 is implemented as a button, a toggle block, or a rotary disc, any of which is exposed on the outer surface of the connecting portion 102. When actuated, the push-actuated release mechanism 112 drives the limiting component 196 inward or rotationally to release the mechanical lock between the comb body 108 and the connecting portion 102.

In one specific embodiment, the comb body 108 is rotatably connected to the connecting portion 102 about a common rotational axis W. The connecting portion 102 is formed with a plug-in hole 148, and the push-actuated release mechanism 112 includes a positioning hole 140. The limiting component 196 includes a positioning shaft 144 and the plug-in section 150. The plug-in section 150 engages with the plug-in hole 148 to restrict motion. The push-actuated release mechanism 112 is movably mounted along the direction of the rotational axis W and is configured in a manner such that, when actuated, the push-actuated release mechanism 112 causes the plug-in section 150 to withdraw from the plug-in hole 148, thereby releasing the comb body 108. Simultaneously, the positioning shaft 144 inserted into the positioning hole 140 remains partially engaged, and the positioning shaft 144 rotates within the positioning hole 140 during operation. The positioning shaft 144 is coaxial with the rotational axis W and serves to stabilize and guide the rotation of the comb body 108 around the connecting portion 102.

This mechanism ensures that the comb body 108 remains securely locked during periods of non-use or transport, thereby preventing unintended activation of the physiotherapy element 194 and preserving device integrity. Only upon deliberate user interaction with the push-actuated release mechanism 112, the locking engagement is overcome to allow rotation to the first or second position as required for physiotherapy or storage.

Referring to FIGS. 6, 8, 9, and 10, the limiting component 196 is structurally composed to ensure secure locking of the comb body 108 relative to the connecting portion 102, and to allow reliable actuation and automatic resetting. The limiting component 196 includes a connecting plate 198, two plug-in sections 150, and the positioning shaft 144. The two plug-in sections 150 are arranged at opposite ends of the connecting plate 198, while the positioning shaft 144 is centrally disposed between them. All three components, the two plug-in sections 150 and the positioning shaft 144, are mounted on the same side of the connecting plate 198, allowing for coordinated motion during actuation and reset.

The limiting component 196 further includes a first axle section 142, which extends from the opposite side of the connecting plate 198. A return spring (not shown) is sleeved around the first axle section 142. One end of the spring is supported against the connecting plate 198, while the opposite end is compressed against internal structural features such as a mounting bracket 134 disposed within the comb body 108. The mounting bracket 134 also includes a second axle section 146, on which the opposite end of the return spring is seated.

When the push-actuated release mechanism 112 gets depressed by an external force, the limiting component 196 is driven inward, toward the mounting bracket 134. This movement compresses the return spring and disengages the plug-in sections 150 from the plug-in hole 148 provided in the connecting portion 102. Once the external force on the push-actuated release mechanism 112 is removed, the energy stored in the compressed return spring causes the limiting component 196 to move in the opposite direction, thereby automatically resetting the plug-in sections 150 into the plug-in hole 148 and re-engaging the lock between the comb body 108 and the connecting portion 102.

In alternative embodiments, instead of using a return spring for resetting, the push-actuated release mechanism 112 is configured for manual resetting, such as by outward pulling of the rotational return, depending on the design of the release mechanism (e.g., toggle, button, or rotary type).

In further embodiments, the plug-in hole 148 is omitted. Instead, a protruding bump or detent is provided on the connecting portion 102, which mechanically blocks or engages with the movement of the plug-in sections 150 of the limiting component 196, thus achieving the same locking effect.

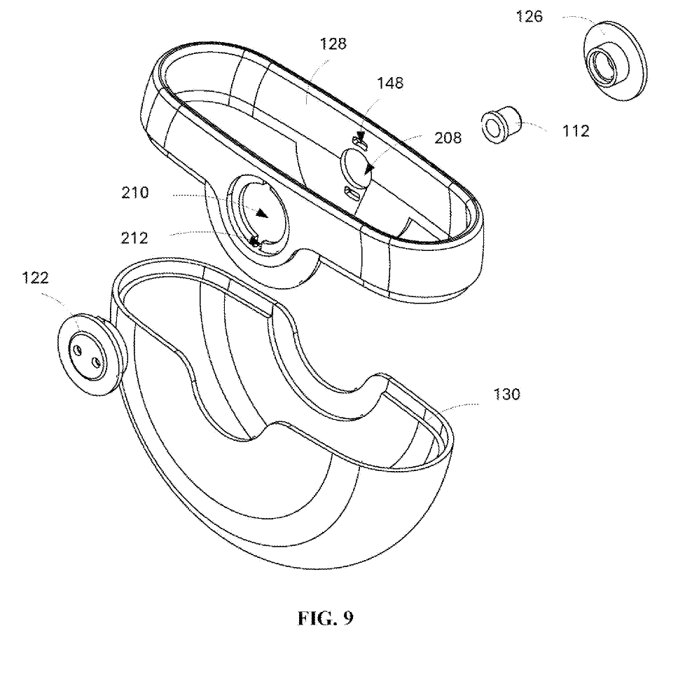

Referring to FIG. 9, in one embodiment, the connecting portion 102 includes a fixing hole 210 on one side and a relinquishing hole 208 on the opposite side. A rotating shaft 122 is mounted through the fixing hole 210, while the push-actuated release mechanism 112 is installed through the relinquishing hole 208. Both holes extend through the storage chamber 132 of the connecting portion 102, enabling access to internal components. The comb body 108 includes a shaft hole 118, which is rotatably coupled with the rotating shaft 122. On the opposite end, the comb body 108 is rotatably coupled to the positioning shaft 144 via the positioning hole 140, thereby providing dual-axis support. This dual connection enhances rotational stability of the comb body 108 during opening and closing between the first and second positions.

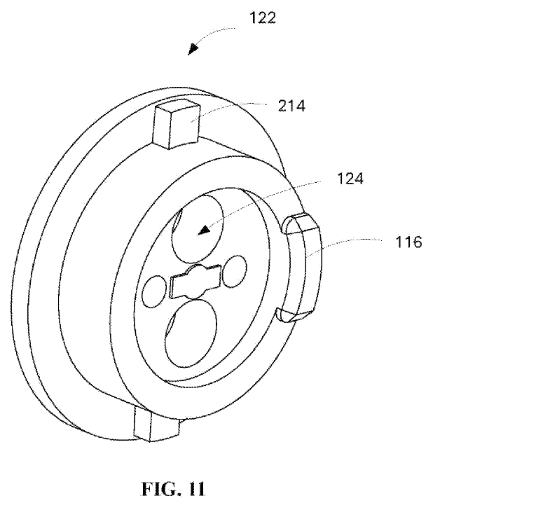

Referring to FIG. 11, in one embodiment of the present disclosure, the first switching element 116 is mounted on the rotating shaft 122, which is installed within the connecting portion 102. The first switching element 116 is positioned eccentrically relative to the central rotational axis of the rotating shaft 122. Optionally, the first switching element 116 is formed as a protrusion on the surface of the rotating shaft 122, such that during rotation, it periodically aligns with the second switching element 117, which is disposed within the comb body 108 and is configured as a travel switch or equivalent contact sensor.

In this configuration, the shaft hole 118 formed in the comb body 108 communicates with the interior cavity of the comb body 108, where a circuit board 136 is housed. The second switching element 117 is fixed to the circuit board 136 and electrically connected thereto. When the comb body 108 is rotated into the first position, the protruding first switching element 116 comes into contact with or approaches the second switching element 117, thereby completing a circuit and enabling activation of the physiotherapy element 194.

The rotating shaft 122 is further provided with one or more mounting through-holes 124, which accommodate charging electrodes. In some embodiments, a pair of mounting through-holes 124 is formed on opposite sides of the rotating shaft 122, each receiving a charging electrode configured to serve as either a positive or negative terminal. These charging electrodes are electrically coupled to the circuit board 136 inside the comb body 108 and are configured to receive power from an external power source for charging an internal battery 202. This structural integration eliminates the need to open the housing of the connecting portion 102 for charging access and avoids additional external connectors.

Referring to FIGS. 9 and 11, the connecting portion 102 includes a limiting groove 212, and the rotating shaft 122 includes at least one limiting protrusion 214 configured to engage with the limiting groove 212. This mechanical coupling restricts undesired rotation of the rotating shaft 122 relative to the connecting portion 102, enhancing rotational stability. Optionally, two limiting protrusions 214 are formed on the rotating shaft 122, angularly spaced at 180 degrees about the central axis of the shaft to provide bilateral locking.

In various embodiments, the switching piece 115 of the physiotherapy comb 100 is provided in different forms. In one configuration, the switching piece 115 operates as a mechanical or micro travel switch, wherein the first switching element 116 is implemented as a protruding contact, and the second switching element 117 is a travel switch mounted to the circuit board 136. In other embodiments, the switching piece 115 is implemented as a proximity-based Hall switch, wherein the first switching element 116 is configured as a magnet and the second switching element 117 as a Hall effect sensor. As the magnet approaches or moves away from the Hall sensor during rotation of the comb body 108, the magnetic field strength sensed by the Hall element changes, triggering an activation or deactivation signal to the circuit board 136 accordingly.

Alternatively, other switching mechanisms may also be employed, such as pressure switches, infrared sensors, or other contactless activation devices, depending on the desired factor and sensitivity requirements of the physiotherapy comb. The physiotherapy element 194 disposed in the comb body 108 includes one or more therapeutic modalities. In one embodiment, the phototherapy lamp 158, incorporating light-emitting diodes (LEDs) or laser selected from red, blue, or violet spectral emitters, is configured to perform light therapy on the scalp. Additionally, the physiotherapy comb incorporates a UV light source, specifically designed for disinfection or sterilization purposes. In another embodiment, the physiotherapy element 194 includes a pair of microcurrent electrodes disposed adjacent to or integrated with the comb teeth 110, which activate upon contact with the skin to deliver low-intensity electrical stimulation. In yet another configuration, the physiotherapy element 194 may include a vibration motor, which transmits oscillatory motion through the comb body 108 to facilitate scalp massage during use. In yet another embodiment, the physiotherapy element 194 may include a piezoelectric element to provide an electro-mechanical stimulus (piezoelectric stimulation) to the soft tissues of the user’s scalp. In another embodiment, the physiotherapy element 194 may include an ultrasonic wave therapy element to provide ultrasonic wave therapy to the user’s scalp to stimulate hair growth and improve scalp health. In yet another embodiment, the physiotherapy element 194 may include a thermal element, such as a heating or cooling element, configured to facilitate hot compression or cold compression therapy for the user’s scalp.

Each of these physiotherapy modalities may be used independently or in combination, depending on the application, and are automatically enabled or disabled based on the positional relationship between the first switching element 116 and the second switching element 117.

Referring to FIGS. 6-8, the comb body 108 is further provided with a liquid reservoir 152 formed within a reservoir housing 138, and a liquid guide column 186 positioned on the same side as the comb teeth 110. The liquid guide column 186 includes at least one fluid passage that provides fluid communication between the liquid reservoir 152 and an external outlet located adjacent to the comb teeth 110. At the distal end of the liquid guide column 186, a roller ball (not shown) is disposed and configured to rotate upon contact with the scalp or hair surface. The rotational motion of the roller ball enables controlled dispensing of the liquid from the liquid reservoir 152 through the fluid passage, allowing application of treatment or conditioning liquid during combing.

During operation, a treatment solution stored within the liquid reservoir 152 flows through the fluid passage and is dispensed onto the user’s scalp or hair while combing. Because the liquid guide column 186 is co-located with the comb teeth 110, the delivery of treatment liquid and combing occur simultaneously, thereby enhancing the therapeutic efficacy and convenience of the physiotherapy comb 100.

In some embodiments, the comb body 108 includes a plurality of liquid guide columns 186, each of the liquid guide column 186 is equipped with its own fluid passage. To facilitate uniform distribution of liquid across these multiple outlets, the comb body 108 further includes an intermediate separating chamber 172, disposed between the liquid reservoir 152 and the outlet fluid passages. In this configuration, the liquid from the liquid reservoir 152 first enters the intermediate separating chamber 172, from which it is evenly directed through a series of fluid passages, each terminating at a corresponding liquid guide column 186. The outlet of each fluid passage is embedded within or adjacent to the comb teeth 110.

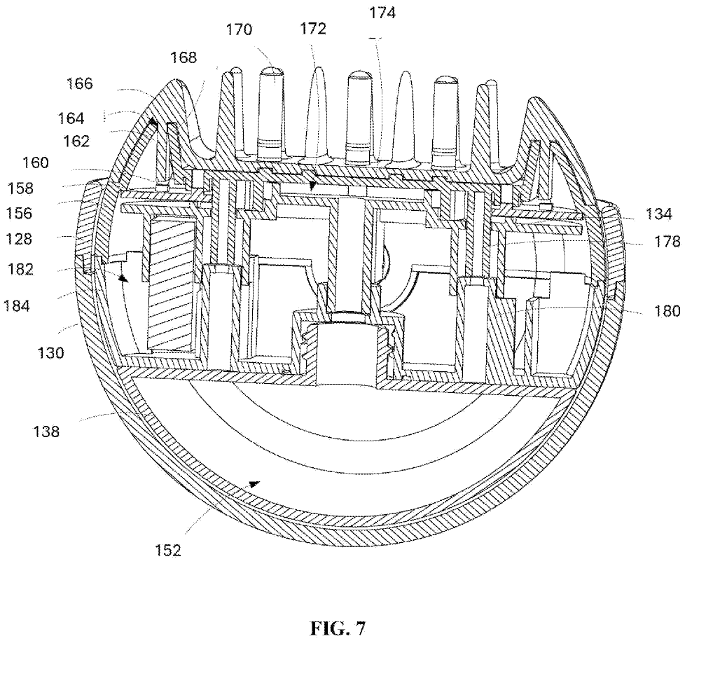

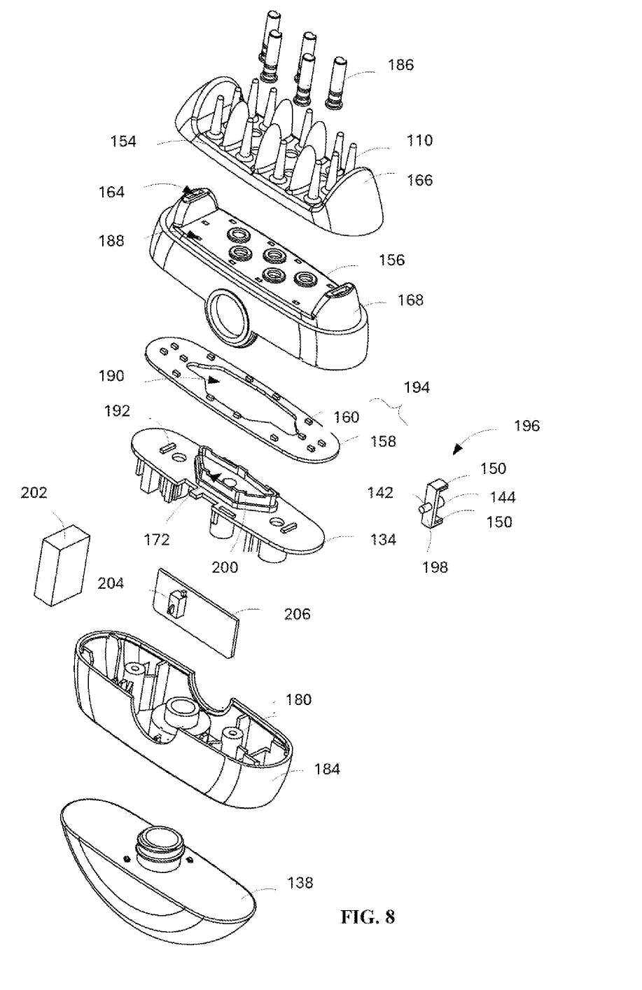

Referring to FIG. 7 and 8, the comb body 108 is constructed from a combination of structural components, including a first casing 156, a second casing 184, and a mounting bracket 134. The first casing 156 and the second casing 184 are assembled to define an installation space 182, within which the physiotherapy element 194 (e.g., a light therapy lamp) and the circuit board 136 are mounted securely on the mounting bracket 134.

The mounting bracket 134 includes a structural rib 200 formed on the side facing the first casing 156. The structural rib 200 encloses the intermediate separating chamber 172, which is sealed by the inner surface of the first casing 156 to prevent leakage. A sealing ring or equivalent sealing structure is positioned around the structural rib 200 to ensure leak-tight integrity of the separating chamber when the casings are assembled.

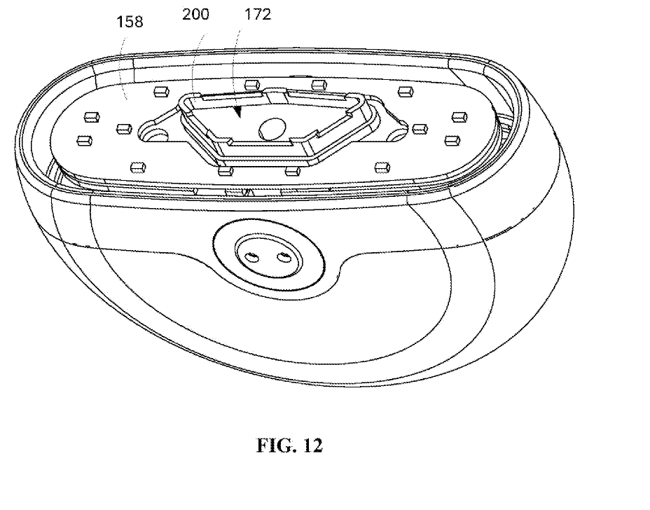

The physiotherapy element 194, when implemented as a light therapy element, comprises a phototherapy lamp 158 with a plurality of light-emitting diodes 160. The phototherapy lamp 158 is centrally provided with a through-hole 190, which is designed to fit over the structural rib 200, thereby enabling precise alignment and mounting of the lamp module over the intermediate separating chamber 172, as illustrated in FIG. 12. This spatial configuration allows the plurality of light-emitting diodes 160 to irradiate through the comb structure, when the comb is in the first position or open position, while maintaining the internal liquid distribution system.

In an embodiment, the phototherapy lamp 158 includes a UV light source. When the comb is in the second position or closed position, the UV light source housed within the phototherapy lamp 158 becomes active and emits light directly onto the dispensing components or the comb teeth 110 and the liquid guide column 186. This setup improves the overall disinfection efficiency while also safeguarding the user's eyes by keeping the UV light contained within the device during operation. As a result, it effectively mitigates contamination risks arising from regular use.

Additionally, the mounting bracket 134 is equipped with a plurality of supporting ribs 192 to structurally support the phototherapy lamp 158 at an elevated position. These ribs create a clearance gap between the phototherapy lamp 158 and the base of the bracket to aid in thermal dissipation during operation of the physiotherapy comb 100. The supporting ribs 192 also serve to minimize installation errors by maintaining the alignment and levelling of the phototherapy lamp 158 during assembly.

The installation space 182 further accommodates other internal components such as the internal battery 202 and the circuit board 136, which are generally inserted into designated slots formed on the side of the mounting bracket 134 facing the second casing 184. These features ensure compact and secure placement of electrical and functional elements within the comb body 108.

Referring to FIG. 7, in an embodiment of the present disclosure, the comb body 108 includes a structural assembly composed of a first casing 156 and a second casing 184, which is configured to enclose an installation space 182. The first casing 156 is provided with one or more first fixing columns 178, each having a corresponding first locking hole. The second casing 184 is provided with one or more second fixing posts 180, each of which includes a second locking hole.

During assembly, the first and second locking holes are aligned along a common axis, allowing a fastener, such as a screw, to pass through and securely couple the first casing 156 and the second casing 184 together. In some embodiments, the second locking hole extends to an outer face of the second casing 184, allowing the fastener to be inserted externally for ease of access and manufacturing.

To ensure precise alignment and avoid interference with other internal components, both the mounting bracket 134 and the phototherapy lamp 158 include avoidance holes that correspond to the positions of the first fixing columns 178 and the second fixing posts 180. These avoidance holes ensure that the structural columns do not obstruct internal installations, while also contributing to positional constraint and stabilization of the mounting bracket 134 and phototherapy lamp 158. When the two casings are locked together, the phototherapy lamp 158 and the mounting bracket 134 are jointly clamped between them, maintaining secure placement of internal components such as the physiotherapy element 194 and circuit board 136.

Both the first casing 156 and the second casing 184 are made from a light-shielding material to prevent stray light leakage and ensure directional emission. The first casing 156 includes a set of light-transmitting apertures 188, each corresponding to a position of a light-emitting diode 160 mounted on the phototherapy lamp 158. These apertures allow therapeutic light, such as red, blue, or violet wavelengths, from the plurality of light-emitting diodes 160 to be emitted outward through the front face of the comb body 108.

The comb body 108 further includes the reservoir housing 138, defining the liquid reservoir 152. In some embodiments, the reservoir housing 138 is disposed on the side of the second casing 184 opposite the first casing 156. A threaded connection is provided for attaching the reservoir housing 138 to the second casing 184. For example, an internal thread is formed within a connection port on the second casing 184, while the reservoir housing 138 is provided with an externally threaded neck that mates securely with the port.

Additionally, the comb body 108 includes a light-transmissive base plate 154 to which the plurality of comb teeth 110 are attached. In one embodiment, the light-transmissive base plate 154 and the comb teeth 110 are formed as an integral, monolithic structure from a transparent or translucent polymer. The light-transmissive base plate 154 is mounted to the outer face of the first casing 156, covering the light-transmitting apertures 188 and permitting light to pass through to the scalp and hair during use.

The light-transmissive base plate 154 includes a first surface and an opposing second surface. On the first surface, the two first convex portions 166 are formed, and the comb teeth 110 are positioned between them. Each first convex portion 166 is hollow, providing light-guiding geometry or structural reinforcement. On the second surface, a corresponding slot is formed for each first convex portion 166, allowing the convex portions to be inserted and stabilized.

The outer surface of the first casing 156 is also provided with a second convex portion 168, which is dimensioned and positioned to insert into the respective slots on the light-transmissive base plate 154. This interlocking configuration prevents undesired movement or shifting of the light-transmissive base plate 154 relative to the first casing 156 during use, thereby maintaining optical alignment and mechanical stability.

In certain embodiments of the present invention, as illustrated in FIG. 7, the second convex portion 168 of the first casing 156 is provided with a jack 164 that opens into the installation space 182 of the comb body 108. A corresponding insert rib 162 is configured to fit within the jack 164 and extends into the installation space 182. The insert rib 293, the light-transmissive base plate 154, the first convex portions 166, and the comb teeth 110 are generally formed as an integral injection-molded component, using a light-transmissive material. This integrated design allows the insert rib 162 to guide therapeutic light emitted from internal light-emitting diodes 160 mounted on the phototherapy lamp 158 directly to the first convex portions 166 and the comb teeth 110.

At the interface of each insert rib 293, the phototherapy lamp 158 is aligned such that a corresponding light-emitting diode 160 is located at the inward-facing end of the rib. This configuration enables direct transmission of light from the internal LEDs to the outer surface of the comb body. As a result, the first convex portions 166 serve as light-diffusing or focusing outlets, increasing the effective phototherapy area during use. In addition, the presence of the insert ribs 162 helps mechanically register and locate the light-transmissive base plate 154 during assembly, enhancing structural stability and alignment accuracy.

The comb teeth 110 are designed with variable geometry to provide different tactile sensations during use. In one configuration, a portion of the comb teeth 110 may have a conical profile, while others may exhibit a flattened or paddle-like structure, enabling diverse combing or scalp massage experiences based on user preference or therapeutic goals.

The light-transmissive base plate 154 is further provided with one or more via holes, each configured to allow passage of the liquid guide column 186. These via holes ensure fluid connectivity between the liquid reservoir 152, the intermediate separating chamber 172, and the dispensing outlets positioned near the comb teeth 110.

Referring to FIG. 9, the connecting portion 102 includes a third shell 130 and a fourth shell 128. In some embodiments, the third shell 130 is formed with a semicircular profile, while the fourth shell 128 is annular and encircles the outer edge of the third shell 130, enclosing the internal components and providing a secure housing structure. The fourth shell 128 is formed with the fixing hole 210 for mounting the rotating shaft 122 and a relinquishing hole 208 through which the push-actuated release mechanism 112 is inserted.

In further embodiments, the connecting portion 102 also includes an annular piece 126, which is generally press-fitted or inserted into the relinquishing hole 208. The annular piece 126 surrounds the push-actuated release mechanism 112 and provides structural support to the push-actuated release mechanism 112 and guidance during its axial or rotational movement. Additionally, the annular piece 126 also serves an aesthetic or decorative purpose, concealing the mechanical interface while enhancing the visual design of the physiotherapy comb 100.

In an exemplary use case, a user prepares to utilize the physiotherapy comb 100 for combined hair grooming and scalp therapy. The physiotherapy comb 100 includes a comb body 108 rotatably connected to a connecting portion 102 via the rotating shaft 122, such that the comb body 108 is movable between a first position and a second position about a defined rotational axis W. Initially, the comb body 108 is in the second position in which the plurality of comb teeth 110 are fully enclosed within the storage chamber 132 defined by the connecting portion 102. In this state, the first switching element 116 is located on the connecting portion 102, and the second switching element 117 is disposed on the comb body 108 that may be spatially separated, thereby maintaining the physiotherapy element 194 in an inactive state.

To activate the physiotherapy comb 100, the user depresses the push-actuated release mechanism 112, which causes the limiting component 196 that includes the positioning shaft 144, the plug-in sections 150, and the connecting plate 198 to disengage from the plug-in hole 148 formed in the connecting portion 102. This disengagement allows the comb body 108 to rotate freely with respect to the connecting portion 102. As the user rotates the comb body 108 from the second position to the first position, the comb teeth 110 extend outward from the storage chamber 132 and become fully exposed for use. At the same time, the first switching element 116, optionally configured as a magnet or eccentric protrusion, comes into proximity or contact with the second switching element 117, which may be implemented as a Hall effect sensor or travel switch. This proximity triggers the second switching element 117 to send a start-up signal to the circuit board 136, which is disposed within the comb body 108.

Upon receiving the activation signal, the circuit board 136 supply power from the internal battery 202 to the physiotherapy element 194, which include one or more of the following modalities, the phototherapy lamp 158 equipped with the plurality of light-emitting diodes 160 (e.g., red, blue, violet LEDs or laser) for delivering phototherapy to the scalp, a pair of microcurrent electrodes, which may be integrated with or positioned adjacent to the comb teeth 110, for delivering mild electrical stimulation upon contact with the user’s scalp, the vibration motor, which may impart soothing vibrational massage through the comb body 108 to enhance blood circulation.

Simultaneously, the user benefits from the liquid dispensing function. The comb body 108 includes the liquid reservoir 152 enclosed within the reservoir housing 138, which stores a therapeutic solution such as hair serum, hair oil, or scalp tonic. The liquid is directed from the liquid reservoir 152 into the intermediate separating chamber 172 formed by the structural rib 200 on the mounting bracket 134. From there, the liquid is evenly distributed through fluid passages to the liquid guide column 186, which is co-located with the comb teeth 110. As the user combs their hair, the liquid exits via guide holes in the liquid guide column 186 and is applied directly to the scalp. The entire assembly of the comb body 108, including the first casing 156, the second casing 184, the mounting bracket 134, and internal electronics, is held together using first fixing columns 178 and the second fixing posts 180, which may be fastened via screw-in locking holes. These structures secure the components within an installation space 182, wherein the phototherapy lamp 158 is mounted with the light-emitting diodes 160 aligned precisely beneath light-transmitting apertures 188 in the first casing 156.

The light-transmissive base plate 154, integrally formed with the comb teeth 110 and the first convex portions 166, overlays the first casing 156 and aligns with the light-transmitting apertures 188. Optional insert ribs 162, also formed as part of the light-transmissive base plate 154, extend into the jack 164 on the first casing 156 and help channel the emitted light outward through the comb teeth 110, thereby increasing the phototherapy surface area. These insert ribs 162 also serve to mechanically register the light-transmissive base plate 154 during assembly.

The user continues to comb their hair, benefitting from the simultaneous delivery of light therapy, microcurrent stimulation, vibrational massage, and liquid application. Upon completion, the user rotates the comb body 108 back into the second position, which automatically increases the distance between the first switching elements 116 and the second switching element 117, thereby deactivating the physiotherapy element 194 via the circuit board 136.

The comb body 108 may re-engage with the plug-in hole 148 via the plug-in sections 150 of the limiting component 196, assisted by a return spring that biases the locking mechanism back into position. This ensures that the comb body 108 remains securely stowed until the next use.

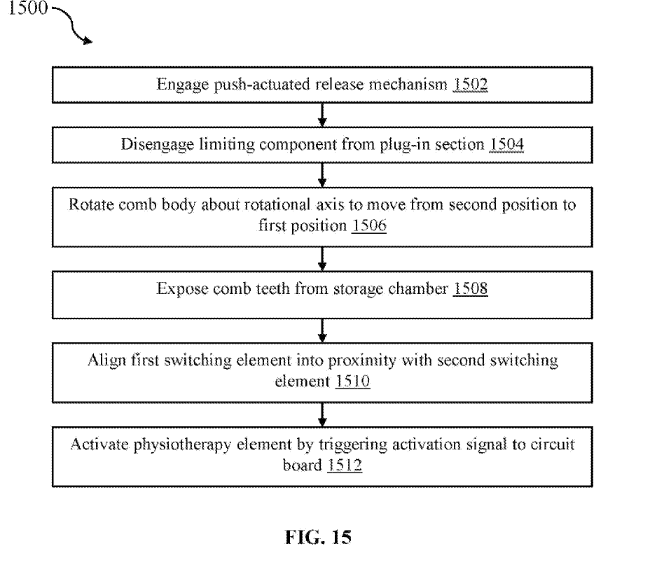

Referring to FIG. 15, in accordance with the present disclosure. At step 1502, a user initiates the process by engaging the push-actuated release mechanism 112, which may be mechanically linked to the limiting component 196. The push-actuated release mechanism 112 may be configured to control the positional locking of the comb body 108 relative to the connecting portion 102. Further, at step 1504, the engagement of the push-actuated release mechanism 112 may result in the disengagement of the limiting component 196 from the plug-in section 150 formed within the connecting portion 102. This disengagement allows relative rotational motion between the comb body 108 and the connecting portion 102. Further, at step 1506, the comb body 108 may be rotated about a rotational axis W from a second position (in which the comb teeth 110 may be retracted or protected) to a first position (in which the comb teeth 110 may be operatively exposed). This rotational motion physically reorients the comb body 108 such that the embedded therapeutic element becomes eligible for activation. Further, at step 1508, the rotation causes the plurality of comb teeth 110 to be exposed from within the storage chamber 132 defined in the connecting portion 102. The exposure of the comb teeth 110 may be coordinated with the operational readiness of the physiotherapy comb 100.

As a result of the rotation, at step 1510, the first switching element 116 may be disposed in the connecting portion 102 may be aligned with the second switching element 117, which may be disposed within the comb body 108. The second switching element 117 may be electrically connected to the circuit board 136 housed within the comb body 108. Further, at step 1512, the alignment between the first switching element 116 and the second switching element 117 may trigger an activation signal to the circuit board 136. In response, the circuit board 136 may initiate activation of the physiotherapy element 194, which may include any of phototherapy element lamp with one or more LEDs or a laser, a piezoelectric element, a vibration element, an ultrasonic wave therapy element, a heating element, a cooling element, a thermal element, or a micro-current element, depending on the configuration.

The physiotherapy comb of the present invention offers a multifunctional grooming solution that combines hair care with advanced therapeutic modalities such as phototherapy, microcurrent stimulation, vibration massage, and liquid dispensing. Its position-sensitive activation mechanism, integrated safety features, and compact modular design ensure user-friendly operation, improved hygiene, and energy efficiency. The physiotherapy comb is highly suitable for industrial-scale production in the personal care and wellness sector, with potential applications in home use, salons, and therapeutic clinics. The innovative features of this device meet the growing demand for multifunctional, smart personal grooming tools, making it a valuable addition to the health and beauty industry.

Thus, the disclosed method and the physiotherapy comb try to overcome the technical problem of accidental or unintended activation of physiotherapy functionalities in conventional combs that rely on manual buttons or switches. Traditional devices often suffer from inadvertent power-on events during storage, transport, or handling, which may lead to battery drain, user discomfort, or inefficient operation. By integrating a mechanically triggered switching mechanism activated solely through intentional rotational movement of the comb body relative to the connecting portion, the present invention ensures controlled activation and deactivation of the physiotherapy element, thereby enhancing usability, safety, and energy efficiency.

As will be appreciated by those skilled in the art, the designs described in the various embodiments discussed above are not routine, or conventional, or well-understood in the art. In light of the above-mentioned advantages and the technical advancements provided by the disclosed method and the physiotherapy comb, the claimed steps as discussed above are not routine, conventional, or well understood in the art, as the claimed steps enable the following solutions to the existing problems in conventional technologies. Further, the claimed steps bring an improvement in the functioning of the device itself, as the claimed steps provide a technical solution to a technical problem.

The specification has described a physiotherapy comb and a method of activating the same. The illustrated steps are set out to explain the exemplary embodiments shown, and it should be anticipated that ongoing technological development will change the manner in which particular functions are performed. These examples are presented herein for the purpose of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

It is intended that the disclosure and examples be considered as exemplary only, with a true scope of disclosed embodiments being indicated by the following claims.

1. A physiotherapy comb comprising:
a connecting portion defining a storage chamber;
a comb body having a plurality of comb teeth and rotatably connected to the connecting portion about a rotational axis, wherein the comb body is movable between a first position and a second position with respect to the connecting portion;
a physiotherapy element disposed within the comb body; and
a circuit board disposed within the comb body and electrically connected to the physiotherapy element,
wherein, when the comb body is moved to the first position, the physiotherapy element is activated.

2. The physiotherapy comb of claim 1, further comprising:
a first switching element disposed within the connecting portion; and
a second switching element disposed within the comb body and electrically connected to the circuit board,
wherein the first switching element and the second switching element are positioned such that, in the first position, the first switching element and the second switching element are aligned to allow activation of the physiotherapy element, the activation being performed upon alignment or via a push-actuated release mechanism, and
wherein, in the second position, the first switching element and the second switching element are spaced apart to disable the physiotherapy element.

3. The physiotherapy comb of claim 1, further comprising:
a push-actuated release mechanism and a limiting component,
wherein the limiting component movably engages with a plug-in section of the connecting portion to restrict rotation of the comb body relative to the connecting portion and is disengaged upon actuation of the push-actuated release mechanism.

4. The physiotherapy comb of claim 1, wherein the plurality of comb teeth is exposed in the first position and retracted into the storage chamber in the second position.

5. The physiotherapy comb of claim 1, wherein the physiotherapy element comprises a light therapy lamp comprising at least one light-emitting diode (LED) selected from red, blue, or violet spectrum emitters.

6. The physiotherapy comb of claim 1, wherein the physiotherapy element comprises a pair of microcurrent electrodes disposed adjacent to or integrated with the plurality of comb teeth.

7. The physiotherapy comb of claim 1, wherein the physiotherapy element comprises a vibration motor configured to generate therapeutic vibrations transmitted through the comb body.

8. The physiotherapy comb of claim 1, further comprising:
a liquid reservoir disposed within a reservoir housing of the comb body and a liquid guide column having at least one fluid passage in fluid communication with the liquid reservoir,
wherein the liquid guide column is positioned adjacent to the plurality of comb teeth.

9. The physiotherapy comb of claim 2, wherein the first switching element comprises a magnet and the second switching element comprises a Hall effect sensor configured to detect alignment of the first switching element and the second switching element for activating the physiotherapy element.

10. The physiotherapy comb of claim 1, wherein the comb body comprises;
a first casing; and
a second casing enclosing an installation space,
wherein the installation space accommodates a mounting bracket, the physiotherapy element, and the circuit board, wherein a light-transmission base plate having integrally formed comb teeth is attached to the first casing.

11. The physiotherapy comb of claim 1, wherein the connecting portion comprises a rotating shaft having a mounting through-hole, and an embedded charging electrode disposed within the mounting through-hole, and
wherein the embedded charging electrode is configured to supply power from an external source to the circuit board.

12. A physiotherapy comb comprising:
a connecting portion defining a storage chamber;
a first switching element disposed within the connecting portion;
a comb body having a plurality of comb teeth and rotatably connected to the connecting portion about a rotational axis, wherein the comb body is movable between a first position and a second position with respect to the connecting portion;
a physiotherapy element disposed within the comb body; and
a second switching element disposed within the comb body and electrically connected to a circuit board,
wherein the first switching element and the second switching element are positioned such that, in the first position, the first switching element and the second switching element are aligned to allow activation of the physiotherapy element, and
wherein, in the second position, the first switching element and the second switching element are spaced apart to disable the physiotherapy element.

13. The physiotherapy comb of claim 12, wherein the plurality of comb teeth is exposed in the first position and retracted into the storage chamber in the second position.

14. The physiotherapy comb of claim 12, wherein the physiotherapy element comprises a light therapy lamp comprising at least one light-emitting diode (LED) selected from red, blue, or violet spectrum emitters.

15. The physiotherapy comb of claim 12, wherein the physiotherapy element comprises a pair of microcurrent electrodes disposed adjacent to or integrated with the plurality of comb teeth.

16. A method of activating a physiotherapy comb via mechanical motion, the method comprising:
rotating a comb body, having a plurality of comb teeth, relative to a connecting portion about a rotational axis, from a second position to a first position; and
aligning a first switching element, disposed within the connecting portion, with a second switching element, disposed within the comb body, to allow activation of a physiotherapy element;
wherein the physiotherapy comb comprises:
the connecting portion defining a storage chamber and comprising the first switching element;
the comb body having the plurality of comb teeth and rotatably connected to the connecting portion about the rotational axis, and movable between the first position and the second position;
the physiotherapy element is disposed within the comb body and configured to perform at least one of phototherapy, ultrasonic wave therapy, piezoelectric stimulation, hot or cold compress therapy, microcurrent stimulation, or vibration therapy;
a circuit board disposed within the comb body and electrically connected to the physiotherapy element; and
the second switching element is electrically connected to the circuit board,
wherein, in the first position, the first switching element and the second switching element are aligned to allow activation of the physiotherapy element, and
wherein, in the second position, the first switching element and the second switching element are spaced apart to disable the physiotherapy element.

17. The method of claim 16, wherein rotating the comb body to the first position further comprises:
exposing the plurality of comb teeth from within the storage chamber for combing hair while activating the physiotherapy element.

18. The method of claim 16, wherein the activation of the physiotherapy element comprises:
energizing a light therapy lamp from a plug-in section of the connecting portion, thereby allowing rotation of the comb body from the second position to the first position.

19. The method of claim 16, further comprising:
engaging a push-actuated release mechanism to disengage a limiting component from a plug-in section of the connecting portion, thereby allowing rotation of the comb body from the second position to the first position.

20. The method of claim 16, wherein the first switching element comprises a magnet and the second switching element comprises a Hall effect sensor configured to detect alignment of the first switching element and the second switching element for activating the physiotherapy element.
